(19) United States
(12) Patent Application Publication
(10) Pub. No.: US 2026/0216380 A1
(43) Pub. Date: Jul. 30, 2026

(54) Method For Producing Ultrasound-Sensitive Drug Carrier For Delivering Hydrophobic Drug, And Ultrasound-Sensitive Drug Carrier Using Same

(71) Applicant: BIOINFRA LIFE SCIENCE INC., Seoul (KR)

(72) Inventors: Dong Hee PARK, Seoul (KR); Jong Ryul PARK, Seoul (KR); Ga Young KIM, Seoul (KR); Jong Ho WON, Seoul (KR); Chul Woo KIM, Seoul (KR)

(21) Appl. No.: 18/880,353

(22) PCT Filed: May 26, 2023
(86) PCT No.: PCT/KR2023/007298

§ 371 Date: Oct. 15, 2025

(30) Foreign Application Priority Data
Jul. 14, 2022 (KR) 10-2022-0086875

(51) Int. Cl. A61K 49/22; A61K 47/24; A61K 47/44

(52) U.S. Cl. CPC A61K 49/225; A61K 47/24; A61K 47/44

(57) ABSTRACT

Disclosed are a method for producing an ultrasound-sensitive drug carrier for delivering a hydrophobic drug, and an ultrasound-sensitive drug carrier produced by using same, the method comprising the steps of: (a) dissolving the hydrophobic drug in a drug-supporting oil to obtain a hydrophobic drug-supported oil; and (b) mixing the hydrophobic drug-supported oil, an inert gas, and a phospholipid, and then performing mechanical mixing at a predetermined revolutions per minute (RPM) to produce the ultrasound-sensitive drug carrier.

### FIELD OF THE DISCLOSURE

The present disclosure relates to a method for producing a drug carrier and the drug carrier using the same; more particularly, to a method for producing an ultrasound-responsive (i.e., ultrasound-sensitive) drug carrier for an effective hydrophobic drug delivery and the ultrasound-responsive drug carrier using the same.

### BACKGROUND OF THE DISCLOSURE

Drug Delivery System (DDS) is a dosage formulation for efficiently delivering an amount of drug required to treat a disease while minimizing side effects of the drug and optimizing an efficacy of the drug.

The DDS may be implemented as transdermal, oral, or intravascular methods depending on drug delivery routes. In addition, the DDS that introduces micro-sized capsules into blood vessels to treat affected areas is receiving attention as a desired treatment technology.

Further, among technologies of the DDS, a technique that can precisely target the drug to the affected areas and a technique that can control the drug release at the affected areas are provided. Therefore, a targeted DDS that uses ultrasound wave and an ultrasound-responsive drug carrier, is gaining more interests as a technology that can solve previous problems.

In particular, there have been attempts to deliver the drug through ligand binding of the drug or a receptor to a membrane of the ultrasound-responsive drug carrier based on a study which shows that the ultrasound-responsive drug carrier used as ultrasound contrast agents can cause cavitation due to ultrasound energy and that the cavitation allows more effective drug delivery into a skin or a cell.

However, since this kind of method binds the drug to the membrane, there is a limitation in that the drug may be lost before the ultrasound-responsive drug carrier arrives at a target site, and thus does not perfectly serve as drug carriers. Also, there is another problem in that this kind of method cannot carry large dosage of the drug.

Therefore, an improvement for solving this problem is required.

### DETAILED EXPLANATION OF THE DISCLOSURE

### Objects of the Invention

It is an object of the present disclosure to solve all the aforementioned problems.

It is another object of the present disclosure to produce an ultrasound-responsive drug carrier (i.e., an ultrasound-sensitive drug carrier) in a form of a phospholipid single layer.

It is still another object of the present disclosure to protect a hydrophobic drug from an external environment by loading the hydrophobic drug into the ultrasound-responsive drug carrier.

It is still another object of the present disclosure to load the hydrophobic drug more than a certain amount into the ultrasound-responsive drug carrier in order to achieve a significant drug effect.

It is still yet another object of the present disclosure to effectively deliver the hydrophobic drug to a target site where ultrasound wave is irradiated by using the high responsive characteristic of the ultrasound-responsive drug carrier.

It is still yet another object of the present disclosure to enable the hydrophobic drug to be delivered to the target site effectively by preventing the hydrophobic drug in the ultrasound-responsive drug carrier from being naturally released during delivery.

It is still yet another object of the present disclosure to produce the ultrasound-responsive drug carrier having a certain size distribution.

### Means of Solving the Problem

In order to accomplish objects above and characteristic effects to be described later of the present disclosure, distinctive structures of the present disclosure are described as follows.

In accordance with one aspect of the present disclosure, there is provided a method of producing an ultrasound-responsive drug carrier for delivering a hydrophobic drug, including steps of: (a) acquiring hydrophobic drug carrying oil by dissolving the hydrophobic drug in oil for carrying drug; and (b) producing the ultrasound-responsive drug carrier by performing a mechanical mixing of the hydrophobic drug carrying oil, inert gas, and phospholipid according to a preset RPM (revolutions per minute).

As one example, the step of (b) includes steps of: (b1) upon a determination of a range of target size of the ultrasound-responsive drug carrier, determining a target RPM corresponding to the range of target size as the preset RPM; and (b2) mixing the hydrophobic drug carrying oil, the inert gas, and the phospholipid, and then performing the mechanical mixing thereof according to the target RPM, to thereby produce the ultrasound-responsive drug carrier.

As one example, the step of (a) includes steps of: (a1) upon a determination of a range of target size of the ultrasound-responsive drug carrier, determining a specific oil candidate for carrying drug, as the oil for carrying drug, having a target viscosity range corresponding to the range of target size by referring to each of viscosity ranges of a plurality of oil candidates for carrying drug one of which includes the oil for carrying drug; and (a2) acquiring the hydrophobic drug carrying oil by dissolving the hydrophobic drug in the specific oil candidate for carrying drug.

As one example, a shell of the ultrasound-responsive drug carrier includes the phospholipid, wherein the inert gas is fixed by being contacted with a first part of an inner surface of the shell of the ultrasound-responsive drug carrier, and wherein the hydrophobic drug carrying oil is contacted with a second part, which is a remaining part of the inner surface of the shell of the ultrasound-responsive drug carrier.

As one example, a shell of the ultrasound-responsive drug carrier includes the phospholipid, and an outer surface of the shell and an inner surface of the shell are hydrophilic and hydrophobic, respectively.

As one example, the step of (a) includes steps of: (a3) acquiring an intermediate hydrophobic drug carrying oil in which a first portion of the hydrophobic drug is dissolved in the oil for carrying drug and a second portion of the hydrophobic drug, which is a remaining portion of the hydrophobic drug, is not dissolved in the oil for carrying drug; and (a4) removing the second portion, which is an insoluble portion of the hydrophobic drug, from the intermediate hydrophobic drug carrying oil by centrifuging the intermediate hydrophobic drug carrying oil, to thereby acquire the hydrophobic drug carrying oil.

As one example, the inert gas includes at least part of perfluoromethane, perfluoroethane, perfluoropropane, perfluorobutane, perfluoropentane, perfluorohexane, perfluoroheptane, perfluorooctane, decafluoropentane, perfluoro (2-methyl-3-pentanone), perfluorotributylamine, perfluoro-15-crown-5-ether, perfluoro-1, 3-dimethylcyclohexane, perfluoromethylcyclopentane, perfluorodecalin, perfluoromethyldecalin, perfluoroperhydrobenzyltetralin, PERFECTA and sulfur hexafluoride.

In accordance with another aspect of the present disclosure, there is provided an ultrasound-responsive drug carrier for delivering a hydrophobic drug, including: hydrophobic drug carrying oil acquired by dissolving the hydrophobic drug in oil for carrying drug; inert gas; and a shell with the hydrophobic drug carrying oil and the inert gas included therein.

As one example, upon a determination of a range of target size of the ultrasound-responsive drug carrier, a target RPM corresponding to the range of target size is determined as the preset RPM; and the ultrasound-responsive drug carrier is produced by mixing the hydrophobic drug carrying oil, the inert gas, and the phospholipid, and then performing the mechanical mixing thereof according to the target RPM.

As one example, upon a determination of a range of target size of the ultrasound-responsive drug carrier, a specific oil candidate for carrying drug having a target viscosity range corresponding to the range of target size is determined as the oil for carrying drug by referring to each of viscosity ranges of a plurality of oil candidates for carrying drug, one of which includes the oil for carrying drug; and the hydrophobic drug carrying oil is acquired by dissolving the hydrophobic drug in the specific oil candidate for carrying drug.

As one example, a shell of the ultrasound-responsive drug carrier includes the phospholipid, wherein the inert gas is fixed by being contacted with a first part of an inner surface of the shell of the ultrasound-responsive drug carrier, and wherein the hydrophobic drug carrying oil is contacted with a second part, which is a remaining part of the inner surface of the shell of the ultrasound-responsive drug carrier.

As one example, a shell of the ultrasound-responsive drug carrier includes phospholipid, and an outer surface of the shell and an inner surface of the shell are hydrophilic and hydrophobic, respectively.

As one example, an intermediate hydrophobic drug carrying oil is acquired such that in the intermediate hydrophobic drug carrying oil a first portion of the hydrophobic drug is dissolved in the oil for carrying drug and a second portion of the hydrophobic drug, which is a remaining portion of the hydrophobic drug, is not dissolved in the oil for carrying drug, and then the second portion, which is an insoluble portion of the hydrophobic drug, is removed from the intermediate hydrophobic drug carrying oil by centrifuging the intermediate hydrophobic drug carrying oil, to thereby acquire the hydrophobic drug carrying oil.

As one example, the inert gas includes at least part of perfluoromethane, perfluoroethane, perfluoropropane, perfluorobutane, perfluoropentane, perfluorohexane, perfluoroheptane, perfluorooctane, decafluoropentane, perfluoro (2-methyl-3-pentanone), perfluorotributylamine, perfluoro-15-crown-5-ether, perfluoro-1, 3-dimethylcyclohexane, perfluoromethylcyclopentane, perfluorodecalin, perfluoromethyldecalin, perfluoroperhydrobenzyltetralin, PERFECTA and sulfur hexafluoride.

### Effects of the Invention

The present disclosure has an effect of producing an ultrasound-responsive drug carrier in the form of the phospholipid single layer.

The present disclosure has another effect of protecting hydrophobic drug from an external environment by loading the hydrophobic drug into the ultrasound-responsive drug carrier.

The present disclosure has yet another effect of loading the hydrophobic drug more than a certain amount into the ultrasound-responsive drug carrier in order to achieve a significant drug effect.

The present disclosure has still yet another effect of effectively delivering the hydrophobic drug to a target site where the ultrasound wave is irradiated by using the high responsive characteristic of the ultrasound-responsive drug carrier.

The present disclosure has still yet another effect of enabling the hydrophobic drug to be delivered to the target site effectively by preventing the hydrophobic drug in the ultrasound-responsive drug carrier from being naturally released during delivery.

The present disclosure has still yet another effect of producing the ultrasound-responsive drug carrier having a certain size distribution.

### BRIEF DESCRIPTION OF THE DRAWINGS

The following drawings to be used to explain example embodiments of the present disclosure are only part of example embodiments of the present disclosure and other drawings can be obtained based on the drawings by those skilled in the art of the present disclosure without inventive work.

The patent or application file contains at least one drawing executed in color. Copies of this patent or patent application publication with color drawings will be provided by the Office upon request and payment of the necessary fee.

FIG. 1 is a drawing schematically illustrating an ultrasound-responsive drug carrier (i.e., an ultrasound-sensitive drug carrier) in accordance with one example embodiment of the present disclosure.

FIG. 2 is a drawing schematically illustrating results of producing the ultrasound-responsive drug carrier according to an RPM of a homogenizer which is set in various ways in accordance with one example embodiment of the present disclosure.

FIG. 3A to 3C are drawings schematically illustrating processes and results of separating the ultrasound-responsive drug carrier of various sizes in accordance with one example embodiment of the present disclosure.

FIG. 4 is a drawing schematically illustrating a cumulative amount of a drug being naturally released from the ultrasound-responsive drug carrier in accordance with one example embodiment of the present disclosure.

FIG. 5 is a drawing schematically illustrating an encapsulation efficacy of hydrophobic drug carrying oil in the ultrasound-responsive drug carrier and a hydrophobic drug loading amount in the ultrasound-responsive drug carrier in accordance with one example embodiment of the present disclosure.

FIGS. 6A to 6C are drawings schematically illustrating results of irradiating ultrasound wave onto the ultrasound-responsive drug carrier in accordance with one example embodiment of the present disclosure.

FIG. 7 is a drawing schematically illustrating results of irradiating the ultrasound wave onto breast cancer cells after delivering the ultrasound-responsive drug carrier to the breast cancer cells in accordance with one example embodiment of the present disclosure.

### DETAILED DESCRIPTION OF THE PREFERRED EMBODIMENTS

Detailed explanation on the present disclosure to be made below refer to attached drawings and diagrams illustrated as specific embodiment examples under which the present disclosure may be implemented to make clear of purposes, technical solutions, and advantages of the present disclosure. These embodiments are described in sufficient detail to enable those skilled in the art to practice the disclosure.

Besides, in the detailed description and claims of the present disclosure, a term “include” and its variations are not intended to exclude other technical features, additions, components of steps. Other objects, benefits, and features of the present disclosure will be revealed to one skilled in the art, partially from the specification and partially from the implementation of the present disclosure. The following examples and drawings will be provided as examples but they are not intended to limit the present disclosure.

Moreover, the present disclosure covers all possible combinations of example embodiments indicated in this specification. It is to be understood that the various embodiments of the present disclosure, although different, are not necessarily mutually exclusive. For example, a particular feature, structure, or characteristic described herein in connection with one embodiment may be implemented within other embodiments without departing from the spirit and scope of the present disclosure. In addition, it is to be understood that the position or arrangement of individual elements within each disclosed embodiment may be modified without departing from the spirit and scope of the present disclosure. The following detailed description is, therefore, not to be taken in a limiting sense, and the scope of the present disclosure is defined only by the appended claims, appropriately interpreted, along with the full range of equivalents to which the claims are entitled. In the drawings, like numerals refer to the same or similar functionality throughout the several views.

To allow those skilled in the art to the present disclosure to be carried out easily, the example embodiments of the present disclosure by referring to attached drawings will be explained in detail as shown below.

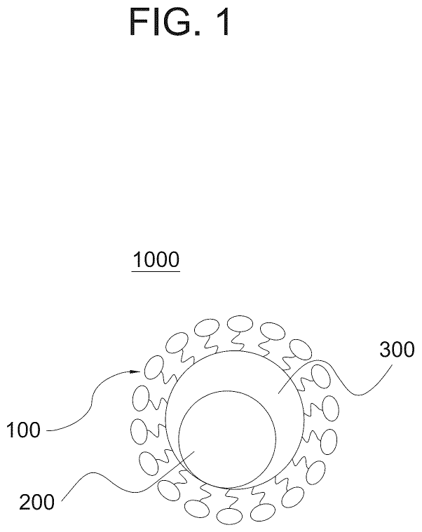

FIG. 1 is a drawing schematically illustrating an ultrasound-responsive drug carrier 1000 for delivering a drug in accordance with one example embodiment of the present disclosure.

By referring to FIG. 1, the ultrasound-responsive drug carrier 1000 according to one example embodiment of the present disclosure may have a shell 100 including phospholipid, hydrophobic drug carrying oil 300 in which a hydrophobic drug is dissolved, and inert gas 200.

For reference, the ultrasound-responsive drug carrier 1000 according to one example embodiment of the present disclosure may be a microbubble having a high responsiveness to ultrasound, but it is not limited thereto.

Herein, the drug to be used may be the hydrophobic drug. Herein, the hydrophobic drug may be a drug that dissolves only in oil and does not dissolve at all in water, but it is not limited thereto, and it may be a drug that dissolves better in oil even if a small amount of the drug may dissolve in water.

As an example, the drug may be the hydrophobic drug including at least part of bendamustine, busulfan, carmustine, chlorambucil, cyclophosphamide, dacarbazine, ifosfamide, melphalan, procarbazine, streptozotocin, temozolomide, asparaginase, capecitabine, cytarabine, 5-fluorouracil, fludarabine, gemcitabine, methotrexate, pemetrexed, raltitrexed, actinomycin D, bleomycin, daunorubicin, epirubicin, idarubicin, mitomycin, mitoxantrone, etoposide, docetaxel, irinotecan, paclitaxel, topotecan, vinblastine, vincristine, vinorelbine, carboplatin, cisplatin, oxaliplatin, alemtuzumab, BCG (Bacillus Calmette Guerin), bevacizumab, cetuximab, denosumab, erlotinib, gefitinib, imatinib, interferon, ipilimumab, lapatinib, panitumumab, rituximab, sunitinib, sorafenib, temsirolimus, trastuzumab, clodronate, ibandronic acid, pamidronate, and zoledronic acid.

Further, the hydrophobic drug carrying oil, i.e., hydrophobic drug-supported oil, may be acquired by dissolving the hydrophobic drug in oil, i.e., the oil for carrying drug.

Moreover, the ultrasound-responsive drug carrier 1000 may be produced by using the hydrophobic drug carrying oil, the inert gas, and the phospholipid.

For reference, the inert gas used to produce the ultrasound-responsive drug carrier 1000 in accordance with one example embodiment of the present disclosure may be gas of perfluorocarbon series (e.g., perfluoromethane, perfluoroethane, perfluoropropane, perfluorobutane, perfluoropentane, perfluorohexane, perfluoroheptane, perfluorooctane, decafluoropentane, perfluoro (2-methyl-3-pentanone), perfluorotributylamine, perfluoro-15-crown-5-ether, perfluoro-1, 3-dimethylcyclohexane, perfluoromethylcyclopentane, perfluorodecalin, perfluoromethyldecalin, perfluoroperhydrobenzyltetralin, PERFECTA), sulfur hexafluoride, and air in accordance with one example embodiment of the present disclosure. For reference, the inert gas included in the ultrasound-responsive drug carrier 1000 may be in liquid form, but it is not limited thereto, and may be in gas form.

In addition, the oil for carrying drug included in the ultrasound-responsive drug carrier 1000 may include at least part of almond oil, apricot oil, avocado oil, canola oil, castor oil, coconut oil, cocoa oil, corn oil, cottonseed oil, linseed oil, medium-chain triglyceride (MCT) oil, palm oil, soybean oil, and sunflower oil, in accordance with one example embodiment of the present disclosure.

Further, according to one example embodiment of the present disclosure, the phospholipid included in the shell of the ultrasound-responsive drug carrier 1000 may include at least part of DPPC, DPPA, DSPE-mPEG-2000, and cholesterol. It is to be noted that the present disclosure is not limited to only these examples, and the phospholipid included in the shell formed on an outer surface of the ultrasound-responsive drug carrier 1000 in accordance with one example embodiment of the present disclosure may include at least part of phosphatidylcholine series (e. g., HSPC, DEPC, DOPC, and DMPC), DMPA-NA, DPPA-Na, DOPA-Na, DSPE, DSPE-mPEG, DSPE-mPEG-2000-Na, DSPE-mPEG-5000-Na, and DSPE-Maleimide PEG-2000-Na.

For example, after putting the hydrophobic drug into the oil (i.e., the oil for carrying drug), an intermediate hydrophobic drug carrying oil may be acquired. Herein, in the intermediate hydrophobic drug carrying oil, a first portion (e.g., a first amount) of the hydrophobic drug is dissolved in the oil for carrying drug and a second portion of the hydrophobic drug, which is a remaining portion of the hydrophobic drug, is not dissolved in the oil for carrying drug. Then, the hydrophobic drug carrying oil may be acquired by removing the second portion, which is an insoluble portion of the hydrophobic drug, from the intermediate hydrophobic drug carrying oil through centrifuging the intermediate hydrophobic drug carrying oil.

For reference, the term “insoluble portion of the hydrophobic drug” may refer to a portion of the hydrophobic drug that is saturated, i.e., no longer soluble in the oil (i.e., the oil for carrying drug), due to the hydrophobic drug being excessively added to the oil for carrying drug to an extent of exceeding a solubility of the hydrophobic drug in the oil for carrying drug.

It is to be noted that the ultrasound-responsive drug carrier 1000 may be produced by performing a mechanical mixing of the hydrophobic drug carrying oil, the inert gas, and the phospholipid according to a preset RPM (revolutions per minute).

A diameter of the ultrasound-responsive drug carrier 1000 produced in accordance with the above process may be 500 nm to 3 um. However, the present invention is not limited thereto. For example, a size of the ultrasound-responsive drug carrier 1000 may be determined by changing a value of RPM for a mechanical mixing or by selecting oil having a different viscosity.

As one example, upon a determination of a range of target size of the ultrasound-responsive drug carrier 1000 and upon a determination of a specific oil candidate for carrying drug (as the oil for carrying drug) having a target viscosity range corresponding to the range of target size by referring to each of viscosity ranges of a plurality of oil candidates for carrying drug, the hydrophobic drug may be dissolved in the specific oil candidate for carrying drug, to thereby acquire the hydrophobic drug carrying oil. Herein, one of the plurality of oil candidates for carrying drug may include the oil for carrying drug. Accordingly, the ultrasound-responsive drug carrier 1000 corresponding to the range of target size may be produced by mixing the hydrophobic drug carrying oil formed as mentioned above, the inert gas, and the phospholipid.

As another example, upon a determination of the range of target size of the ultrasound-responsive drug carrier 1000 and upon a determination of a target RPM corresponding to the range of target size (as the preset RPM), the hydrophobic drug carrying oil, the inert gas, and the phospholipid are mixed, and then the mechanical mixing may be performed on the mixture of the hydrophobic drug carrying oil, the inert gas, and the phospholipid according to the target RPM, to thereby produce the ultrasound-responsive drug carrier 1000 corresponding to the range of target size.

Meanwhile, by referring back to FIG. 1, the shell 100 formed on the outer surface of the ultrasound-responsive drug carrier 1000 is in a form of a phospholipid single layer.

For reference, a head of the phospholipid has a hydrophilic property and a tail thereof has a hydrophobic property, therefore, an inside of the ultrasound-responsive drug carrier 1000 with the shell of the phospholipid single layer as illustrated in FIG. 1 has the hydrophobic property.

Conventionally, it was not possible to load the hydrophobic drug into the ultrasound-responsive drug carrier with a shell formed with a phospholipid bilayer because both surfaces of the shell (i.e., an inner surface and the outer surface) were hydrophilic.

On the other hand, according to one example embodiment of the present disclosure, as illustrated in FIG. 1, there is an advantage of the ultrasound-responsive drug carrier 1000 (with the shell formed as the phospholipid single layer) being able to load hydrophobic substances (e.g., the hydrophobic drug carrying oil) in large quantities inside the ultrasound-responsive drug carrier 1000.

Meanwhile, according to one embodiment of the present invention, instead of loading the hydrophobic drug as-is in the ultrasound-responsive drug carrier 1000, the hydrophobic drug is dissolved in the oil for carrying drug to obtain the hydrophobic drug carrying oil, and then the hydrophobic drug carrying oil can be loaded in the ultrasound-responsive drug Carrier 1000.

If the hydrophobic drug as-is is loaded in the ultrasound-responsive drug carrier 1000 without dissolving the hydrophobic drug in the oil, the hydrophobic drug loaded in this way may crystallize. But, if the ultrasound wave is irradiated onto the ultrasound-responsive drug carrier in which the crystallized hydrophobic drug is loaded, there may be a problem of an uneven releasement of the hydrophobic drug.

Therefore, according to one example embodiment of the present disclosure, after the hydrophobic drug is dissolved in the oil, the ultrasound-responsive drug carrier 1000 is produced by performing the mechanically mixing of a vial in which (i) the hydrophobic drug carrying oil, (ii) the inert gas, and (iii) an aqueous solution of the phospholipid are mixed.

Meanwhile, below, a process for producing the ultrasound-responsive drug carrier 1000 in accordance with one example embodiment of the present disclosure by using a specific hydrophobic drug (e.g., the paclitaxel), a specific oil for carrying drug (e.g., the MCT oil), a specific inert gas (e.g., the perfluorohexane), and a specific phospholipid (e.g., the DPPC, the DPPA, the cholesterol, etc.) will be described.

It is to be noted that the process for producing the ultrasound-responsive drug carrier 1000 described below is only an example for help with understanding the present invention, but the present invention is not limited thereto, and a person skilled in the art will easily understand a method for producing the ultrasound-responsive drug carrier 1000 according to the present invention by using a combination of other hydrophobic drugs, other oils for carrying drug, other inert gases, and other phospholipids described above without excessive efforts.

For reference, the ultrasound-responsive drug carrier 1000 described below may be, but is not limited to, ultrasound-responsive microbubbles that have high responsiveness to the ultrasound wave.

As an example, 15 mg of paclitaxel may be added to a 20 mL microtube. Afterwards, 10 ml of the MCT oil may be added to the microtube.

Next, the paclitaxel contained in the microtube may be dissolved in the MCT oil by using an ultrasonic bath at 40 degrees Celsius for about 2 hours.

Further, by centrifuging the mixture in the microtube at a speed of 14,000 RPM for 20 minutes using a centrifuge, the paclitaxel (in the powdered form) that is not dissolved in the MCT oil may be removed.

Meanwhile, (i) 200 mg of 1,2-Dipalmitoyl-sn-glycerol-3-phosphocholine (DPPC), (ii) 10 mg of diphenylphosphoryl azide (DPPA), (iii) 35 mg of cholesterol alc, and (iv) 10 mg of DSPE-PEG (2000) may be dispersed in 30 mL of propylene glycol and then ultrasonicated by using an ultrasonic bath at 40° C. for about 1 hour. Afterwards, 70 mL of triple-distilled water is added thereto and then ultrasonicated by using the ultrasonic bath at 40° C. for about 1 hour to obtain a phospholipid aqueous solution to be used for manufacturing the ultrasound-responsive drug carrier 1000. For reference, the refrigerated storage temperature for the phospholipid aqueous solution may be 4 degrees Celsius.

Then, (i) 1 mL of the MCT oil with the paclitaxel dissolved therein at a concentration of 1.5 mg/ml, (ii) 18 mL of the phospholipid aqueous solution, (iii) 1 ml of perfluorohexane may be added to a 30 mL glass vial.

For reference, each amount of the MCT oil, the phospholipid aqueous solution, and the perfluorohexane described above may be only examples, and the present disclosure is not limited thereto.

For example, (i) 2 mL of the MCT oil with the paclitaxel dissolved therein (having the concentration of paclitaxel/MCT oil: 1.5 mg/mL), 16 mL of the phospholipid aqueous solution, and 2 mL of perfluorohexane may be added to a 30 mL glass vial, or (ii) 4 mL of the MCT oil containing paclitaxel dissolved therein (having the concentration of paclitaxel/MCT oil: 1.5 mg/mL), 12 mL of the phospholipid aqueous solution, and 4 mL of the perfluorohexane may be added to the 30 mL glass vial.

And, an emulsion (i.e., ultrasound-responsive drug carrier 1000) may be produced by the mechanical mixing of (i) the phospholipid aqueous solution, (ii) the MCT oil with the paclitaxel dissolved therein, and (iii) the perfluorohexane, that are introduced into the glass vial. For reference, a solution with the ultrasound-responsive drug carrier 1000 may be produced by the mechanical mixing of (i) 4500 rpm for 45 seconds when using a vial mixer, or (ii) 35,000 rpm for 5 minutes when using a homogenizer.

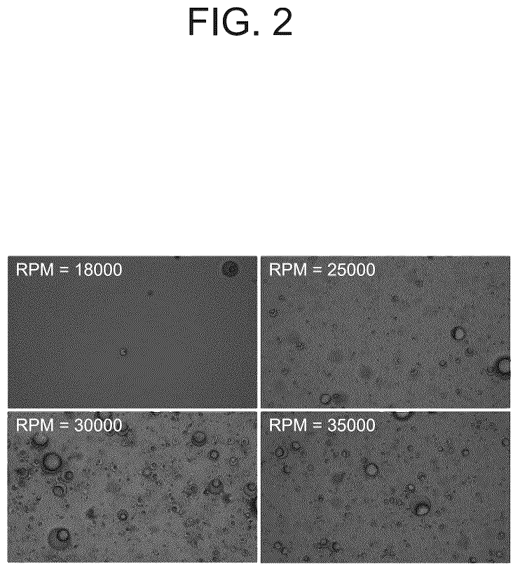

For reference, FIG. 2 illustrates various examples of the ultrasound-responsive drug carrier 1000 produced according to various RPMs.

By referring to FIG. 2, it can be seen that when an RPM of the homogenizer was set to 18,000, almost no ultrasound-responsive drug carrier 1000 was produced. And when the RPM of the homogenizer was set to 25,000, a small amount of the ultrasound-responsive drug carrier 1000 was produced. In addition, when the RPM of the homogenizer was set to 30,000, a relatively large amount of the ultrasound-responsive drug carrier 1000 was produced. And when the RPM of the homogenizer was set to 35,000, a relatively larger amount of the ultrasound-responsive drug carrier 1000 having the target size was produced compared to other cases.

Meanwhile, in accordance with one example embodiment of the present disclosure, in order to further increase a drug delivery effect, some of the ultrasound-responsive drug carriers (except for the ultrasound-responsive drug carriers with a specific size (e. g., 500 nm to 3 um)) may be removed from the ultrasound-responsive drug carriers produced in various sizes (e.g., 100 nm to 10 um).

For example, after performing the mechanical mixing by setting the RPM of the homogenizer to 35,000, 40 ml of distilled water may be added to 20 mL of a solution with the ultrasound-responsive drug carrier 1000 in various sizes, and then the entire solution may be introduced into a 100 mL syringe.

Then, after the syringe is refrigerated at 4 degrees Celsius in atmospheric pressure (e.g., 1 atm) for 1 hour, the ultrasound-responsive drug carriers 1000 with excessively large sizes may be precipitated at the bottom of the syringe due to gravity, therefore, a solution or precipitates located at a bottom of the syringe may be removed. It is to be noted that the volume of the solution or the precipitates removed may be less than 10% of a total volume of the entire solution contained in the syringe.

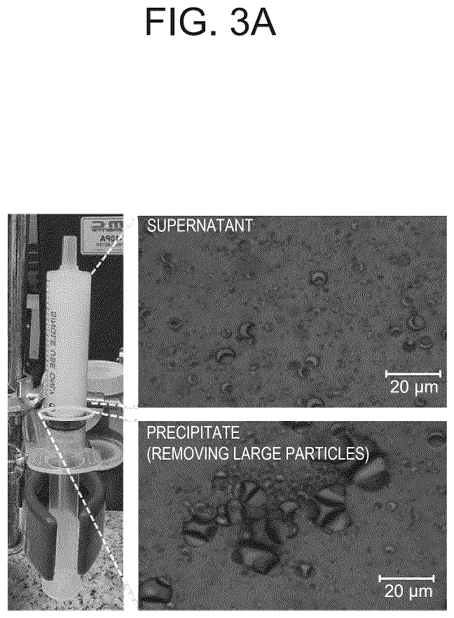

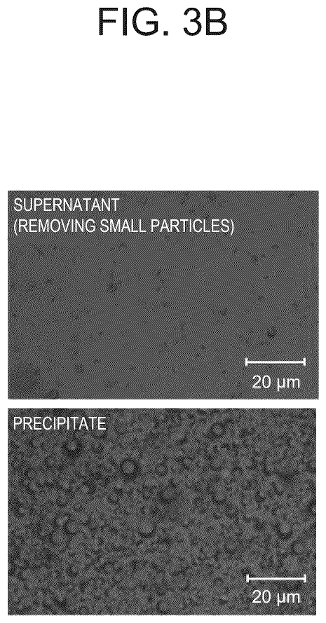

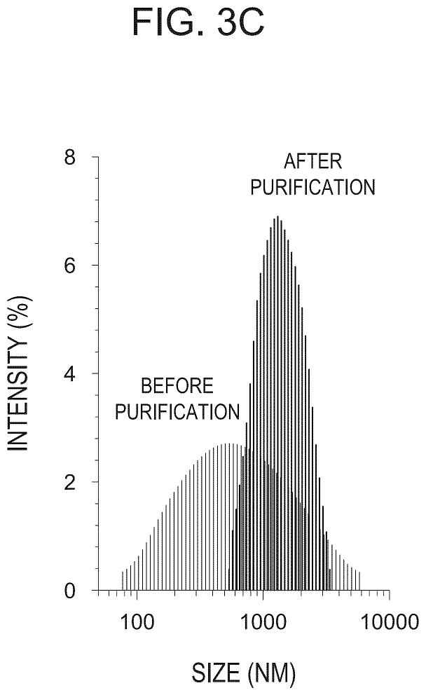

Referring to FIG. 3A, it can be seen that the ultrasound-responsive drug carrier 1000 with the excessively large sizes (as the precipitates) and the remaining supernatant are separated by gravity.

Then, by centrifuging a solution remaining in the syringe at a speed of 2,000 RPM for 20 minutes using a centrifuge, the ultrasound-responsive drug carrier 1000 not precipitated but located in an upper layer (i.e., the ultrasound-responsive drug carriers 1000 with very small sizes) may be removed.

Referring to FIG. 3B, it can be seen that the ultrasound-responsive drug carriers 1000 of a certain range of size (as precipitates) and a remaining supernatant (i.e., the ultrasound-responsive drug carrier 1000 with the very small sizes) are separated by centrifugation.

In addition, by referring to FIGS. 3A and 3B, it can be seen that an interior of the ultrasound-responsive drug carrier 1000 according to one example embodiment of the present disclosure is partitioned such that one of the partitioned interior thereof has a crescent shape.

In particular, it can be seen that the interior of the ultrasound-responsive drug carrier 1000 is partitioned into (i) a space in the crescent shape occupied by the hydrophobic drug carrying oil, and (ii) a remaining space occupied by the inert gas.

Further, by referring to FIGS. 3A and 3B, it can be seen that the inert gas is fixed at a specific location inside the ultrasound-responsive drug carrier 1000.

As one example, in accordance with one example embodiment of the present disclosure, the inert gas may be fixed by being contacted with a first part of the inner surface of the shell of the ultrasound-responsive drug carrier 1000, and the hydrophobic drug carrying oil may be contacted with a second part, which is a remaining part of the inner surface of the shell of the ultrasound-responsive drug carrier 1000.

If it is assumed that the inert gas is not fixed at the specific location inside the ultrasound-responsive drug carrier 1000 and that the inert gas flows freely inside the ultrasound-responsive drug carrier 1000, it would not have exhibited the crescent shape as shown in FIGS. 3A and 3B.

In this way, when the inert gas is fixed to the specific location inside the ultrasound-responsive drug carrier 1000, an alignment direction of the ultrasound-responsive drug carrier 1000 may be determined according to a weight of the inert gas. Therefore, a more effective cavitation effect may be generated by irradiating the ultrasound wave while considering the alignment direction of the ultrasound-responsive drug carrier 1000, thereby allowing the hydrophobic drug to be effectively delivered to a target site.

In addition, by referring to FIG. 3C, it can be seen that (i) if a process of the above example was not performed, only the ultrasound-responsive drug carrier 1000 with sizes of from 50 nm to 6 um can be obtained, whereas (ii) when the process of the above example was performed, the ultrasound-responsive drug carrier 1000 having uniform sizes of from 500 nm to 3 um can be obtained.

Meanwhile, as described above, after acquiring the hydrophobic drug carrying oil by dissolving the hydrophobic drug in the oil, the hydrophobic drug carrying oil may be loaded in the ultrasound-responsive drug carrier to thereby produce the ultrasound-responsive drug carrier 1000 that is stable.

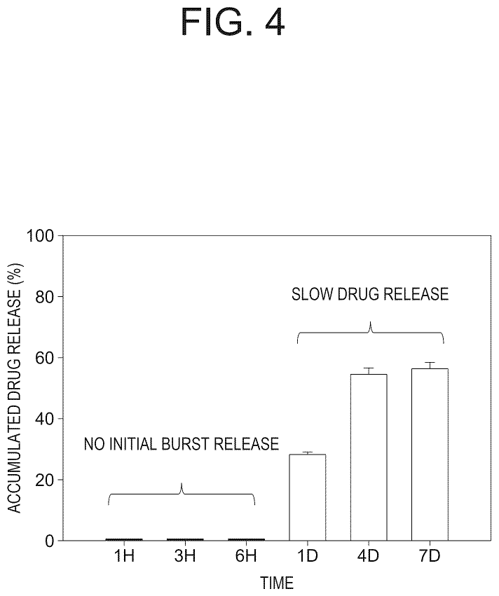

In regard to the above, FIG. 4 illustrates a cumulative amount of the drug being naturally released from the ultrasound-responsive drug carrier 1000 in accordance with one example embodiment of the present disclosure.

It can be seen from FIG. 4 that no drug is naturally released even after 1 hour, 3 hours, and 6 hours have passed from a time of producing the ultrasound-responsive drug carrier 1000 with the hydrophobic drug loaded therein. In addition, it can be seen that even when 1 day has passed from the time of producing the ultrasound-responsive drug carrier 1000 with the hydrophobic drug loaded therein, only about 30% of the drug among a total drug loaded in the ultrasound-responsive drug carrier 1000 was released. Further, 60% of the total drug was released only after 4 to 7 days have passed from the time of producing the ultrasound-responsive drug carrier 1000.

Through this, it can be seen that the ultrasound-responsive drug carrier 1000 produced according to one example embodiment of the present disclosure can load the hydrophobic drug very stably.

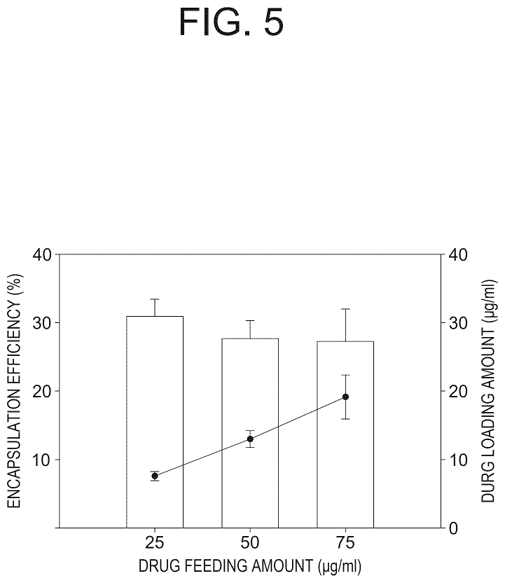

In addition, FIG. 5 is a drawing schematically illustrating (i) an encapsulation efficacy of the hydrophobic drug carrying oil in the ultrasound-responsive drug carrier 1000 and (ii) the hydrophobic drug loading amount in the ultrasound-responsive drug carrier 1000 in accordance with one example embodiment of the present disclosure.

For reference, a horizontal axis of the FIG. 5 represents a concentration of the hydrophobic drug dissolved in the oil for carrying drug, and a left vertical axis of the FIG. 5 represents an encapsulation efficacy of the hydrophobic drug carrying oil in the ultrasound-responsive drug carrier 1000, while a right vertical axis of the FIG. 5 represents the hydrophobic drug loading amount in the ultrasound-responsive drug carrier 1000.

Referring to FIG. 5, it can be seen that the encapsulation efficacy of the hydrophobic drug carrying oil in the ultrasound-responsive drug carrier 1000 according to one example embodiment of the present disclosure stays stable at approximately 30% regardless of the concentration (e.g., 25 ug/mL, 50 ug/mL, and 75 ug/mL) of the hydrophobic drug dissolved in the oil.

Further, by referring to FIG. 5, it can be seen that as the concentration of the hydrophobic drug dissolved in the oil increases respectively up to 25 ug/mL, 50 ug/mL, and 75 ug/mL, the loading amount of the hydrophobic drug loaded into the ultrasound-responsive drug carrier 1000 also respectively increases proportionally (e.g., 10 ug/mL, 15 ug/mL, and 20 ug/mL).

Through this, it can be seen that the loading amount of the hydrophobic drug loaded into the ultrasound-responsive drug carrier 1000 produced according to one example embodiment of the present disclosure may be controlled by controlling the amount of the hydrophobic drug dissolved in the hydrophobic drug carrying oil.

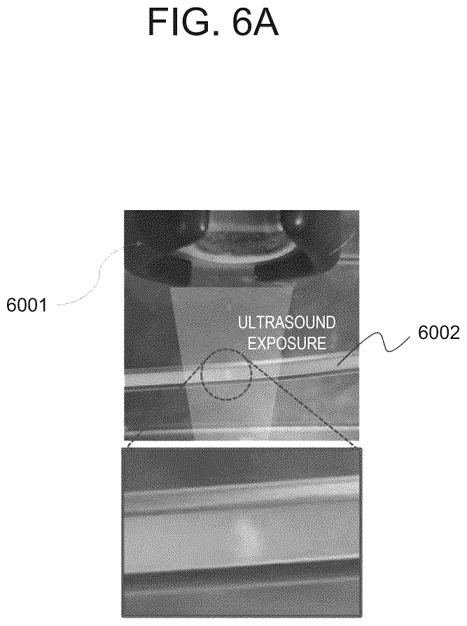

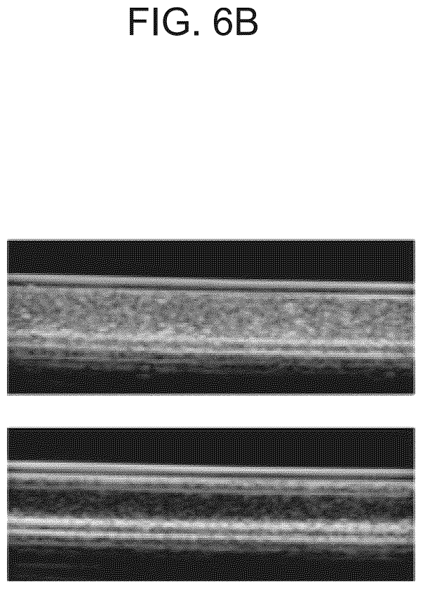

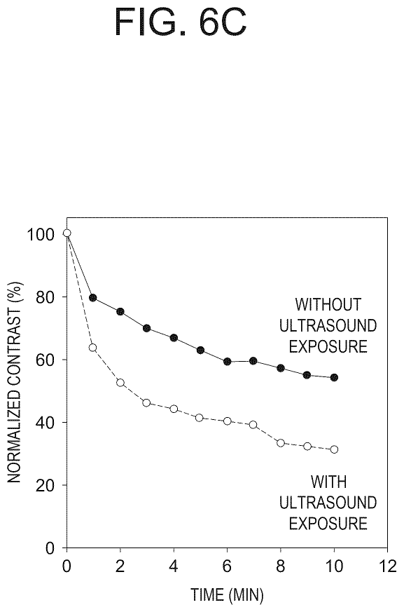

Meanwhile, FIGS. 6A to 6C schematically illustrate observations of irradiating the ultrasound wave onto the ultrasound-responsive drug carrier 1000 for checking responsiveness of the ultrasound-responsive drug carrier 1000 to ultrasound energy in accordance with one example embodiment of the present disclosure.

First, by referring to FIG. 6A, it can be seen that a brightness of a region where the ultrasound-responsive drug carrier 1000 is located is brighter than other areas when the ultrasound wave is irradiated onto the ultrasound-responsive drug carrier 1000 by using an ultrasound transducer 6001, on condition that the ultrasound-responsive drug carrier 1000 is introduced into a silicone tube 6002. Therefore, it can be understood that the ultrasound-responsive drug carrier 1000 according to one example embodiment of the present disclosure is highly responsive to the ultrasound energy.

Further, FIG. 6B illustrates (i) an image of the silicone tube 6002 photographed before irradiating the ultrasound wave onto the ultrasound-responsive drug carrier 1000 and (ii) an image of the silicone tube 6002 photographed after irradiating the ultrasound wave onto the ultrasound-responsive drug carrier 1000 for 10 minutes.

By referring to an upper image of FIG. 6B, it can be seen that an inside of the silicone tube was bright before being irradiated with the ultrasound wave, however, by referring to a lower image of FIG. 6B, it can be seen that the inside of the silicone tube became dark after being irradiated with the ultrasound wave for 10 minutes. This is because, when the ultrasound wave is irradiated onto the ultrasound-responsive drug carrier 1000 that is highly responsive to the ultrasound energy, the ultrasound-responsive drug carrier 1000 may be destroyed by a cavitation effect, destroyed particles may be condensed and the condensed particles may precipitate, thereby darkening the inside of the silicone tube.

Also, FIG. 6C illustrates (i) a brightness of an image of the ultrasound-responsive drug carrier 1000 irradiated with the ultrasound wave and (ii) a brightness of an image of the ultrasound-responsive drug carrier 1000 having not been irradiated with the ultrasound wave. FIG. 6C illustrates that the brightness of the image for the former case is significantly lower than the brightness of the image for the latter case. Through this, it can be understood that the ultrasound-responsive drug carrier 1000 according to one example embodiment of the present disclosure shows high responsiveness to the ultrasound energy.

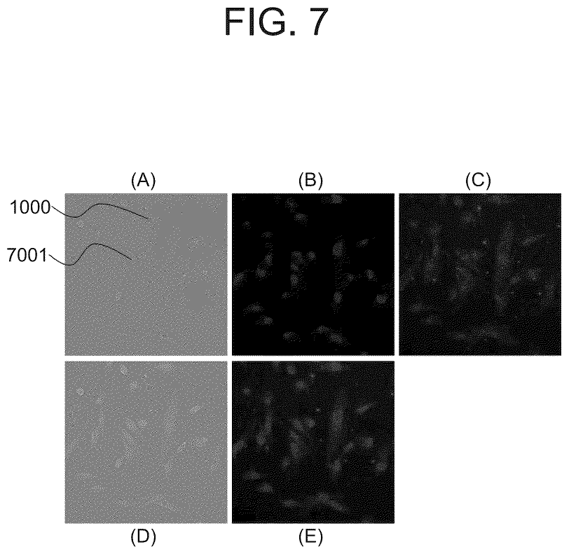

Meanwhile, FIG. 7 is a drawing schematically illustrating results of irradiating the ultrasound wave onto breast cancer cells by using a confocal microscope at different wavelengths of light after delivering the ultrasound-responsive drug carrier to the breast cancer cells in accordance with one example embodiment of the present disclosure.

For reference, in order to easily confirm whether the ultrasound-responsive drug carrier 1000 or the drug loaded therein is effectively introduced into cells, a fluorescent Nile red was loaded into the ultrasound-responsive drug delivery system in place of the hydrophobic drug.

(A) of FIG. 7 shows a result of observing the drug delivery effect in a visible light range. By referring to (A) of FIG. 7, it can be seen that the Nile red and/or the ultrasound-responsive drug carrier 1000 was introduced into the breast cancer cells 7001. This can be understood more clearly through (B) to (E) of FIG. 7.

(B) of FIG. 7 shows a result of observing the nuclei of the breast cancer cells under a wavelength range of 300 nm to 600 nm after dyeing them using a Hoechst stain 3342 (herein, the wavelength range of 300 nm to 600 nm corresponds to an excitation wavelength 350 nm and an emission wavelength 461 nm of the Hoechst stain 3342). Therefore, locations of the nuclei of the breast cancer cells can be seen through the area marked in blue.

In addition, (C) of FIG. 7 illustrates a result of observing the breast cancer cells under a wavelength range of 400 nm to 700 nm (which corresponds to an excitation wavelength 540 nm and an emission wavelength 660 nm of the Nile red), and the area where Nile red exists can be confirmed through the part marked in red.

Moreover, (D) of FIG. 7 is an image acquired by merging the results shown in (A) to (C) of FIG. 7 while (E) of FIG. 7 is an image acquired by merging the results shown in (B) and (C) of FIG. 7. Therefore, by referring to (D) and (E) of FIG. 7, it can be seen that the ultrasound-responsive drug carrier 1000 and/or Nile red loaded therein can be effectively introduced into the interior of the breast cancer cells (particularly, into cytoplasm).

As seen above, the present disclosure has been explained by specific matters such as detailed components, limited embodiments, and drawings. They have been provided only to help more general understanding of the present disclosure. It, however, will be understood by those skilled in the art that various changes and modification may be made from the description without departing from the spirit and scope of the disclosure as defined in the following claims.

Accordingly, the thought of the present disclosure must not be confined to the explained embodiments, and the following patent claims as well as everything including variations equal or equivalent to the patent claims pertain to the category of the thought of the present disclosure.

What is claimed is:

1. A method of producing an ultrasound-responsive drug carrier for delivering a hydrophobic drug, comprising steps of:
(a) acquiring hydrophobic drug carrying oil by dissolving the hydrophobic drug in oil for carrying drug; and
(b) producing the ultrasound-responsive drug carrier by performing a mechanical mixing of the hydrophobic drug carrying oil, inert gas, and phospholipid according to a preset RPM (revolutions per minute).

2. The method of claim 1, wherein the step of (b) includes steps of:
(b1) upon a determination of a range of target size of the ultrasound-responsive drug carrier, determining a target RPM corresponding to the range of target size as the preset RPM; and
(b2) mixing the hydrophobic drug carrying oil, the inert gas, and the phospholipid, and then performing the mechanical mixing thereof according to the target RPM, to thereby produce the ultrasound-responsive drug carrier.

3. The method of claim 1, wherein the step of (a) includes steps of:
(a1) upon a determination of a range of target size of the ultrasound-responsive drug carrier, determining a specific oil candidate for carrying drug, as the oil for carrying drug, having a target viscosity range corresponding to the range of target size by referring to each of viscosity ranges of a plurality of oil candidates for carrying drug one of which includes the oil for carrying drug; and
(a2) acquiring the hydrophobic drug carrying oil by dissolving the hydrophobic drug in the specific oil candidate for carrying drug.

4. The method of claim 1, wherein a shell of the ultrasound-responsive drug carrier includes the phospholipid, wherein the inert gas is fixed by being contacted with a first part of an inner surface of the shell of the ultrasound-responsive drug carrier, and wherein the hydrophobic drug carrying oil is contacted with a second part, which is a remaining part of the inner surface of the shell of the ultrasound-responsive drug carrier.

5. The method of claim 1, wherein a shell of the ultrasound-responsive drug carrier includes the phospholipid, and an outer surface of the shell and an inner surface of the shell are hydrophilic and hydrophobic, respectively.

6. The method of claim 1, wherein the step of (a)
(a3) acquiring an intermediate hydrophobic drug carrying oil in which a first portion of the hydrophobic drug is dissolved in the oil for carrying drug and a second portion of the hydrophobic drug, which is a remaining portion of the hydrophobic drug, is not dissolved in the oil for carrying drug; and
(a4) removing the second portion, which is an insoluble portion of the hydrophobic drug, from the intermediate hydrophobic drug carrying oil by centrifuging the intermediate hydrophobic drug carrying oil, to thereby acquire the hydrophobic drug carrying oil.

7. The method of claim 1, wherein the inert gas includes at least part of perfluoromethane, perfluoroethane, perfluoropropane, perfluorobutane, perfluoropentane, perfluorohexane, perfluoroheptane, perfluorooctane, decafluoropentane, perfluoro (2-methyl-3-pentanone), perfluorotributylamine, perfluoro-15-crown-5-ether, perfluoro-1, 3-dimethylcyclohexane, perfluoromethylcyclopentane, perfluorodecalin, perfluoromethyldecalin, perfluoroperhydrobenzyltetralin, PERFECTA and sulfur hexafluoride.

8. An ultrasound-responsive drug carrier for delivering a hydrophobic drug, comprising:
hydrophobic drug carrying oil acquired by dissolving the hydrophobic drug in oil for carrying drug;
inert gas; and
a shell with the hydrophobic drug carrying oil and the inert gas included therein.

9. The ultrasound-responsive drug carrier of claim 8, wherein, upon a determination of a range of target size of the ultrasound-responsive drug carrier, a target RPM corresponding to the range of target size is determined as the preset RPM; and the ultrasound-responsive drug carrier is produced by mixing the hydrophobic drug carrying oil, the inert gas, and the phospholipid, and then performing the mechanical mixing thereof according to the target RPM.

10. The ultrasound-responsive drug carrier of claim 8, wherein, upon a determination of a range of target size of the ultrasound-responsive drug carrier, a specific oil candidate for carrying drug having a target viscosity range corresponding to the range of target size is determined as the oil for carrying drug by referring to each of viscosity ranges of a plurality of oil candidates for carrying drug, one of which includes the oil for carrying drug; and the hydrophobic drug carrying oil is acquired by dissolving the hydrophobic drug in the specific oil candidate for carrying drug.

11. The ultrasound-responsive drug carrier of claim 8, wherein a shell of the ultrasound-responsive drug carrier includes the phospholipid, wherein the inert gas is fixed by being contacted with a first part of an inner surface of the shell of the ultrasound-responsive drug carrier, and wherein the hydrophobic drug carrying oil is contacted with a second part, which is a remaining part of the inner surface of the shell of the ultrasound-responsive drug carrier.

12. The ultrasound-responsive drug carrier of claim 8, wherein a shell of the ultrasound-responsive drug carrier includes phospholipid, and an outer surface of the shell and an inner surface of the shell are hydrophilic and hydrophobic, respectively.

13. The ultrasound-responsive drug carrier of claim 8, wherein an intermediate hydrophobic drug carrying oil is acquired such that in the intermediate hydrophobic drug carrying oil a first portion of the hydrophobic drug is dissolved in the oil for carrying drug and a second portion of the hydrophobic drug, which is a remaining portion of the hydrophobic drug, is not dissolved in the oil for carrying drug, and then the second portion, which is an insoluble portion of the hydrophobic drug, is removed from the intermediate hydrophobic drug carrying oil by centrifuging the intermediate hydrophobic drug carrying oil, to thereby acquire the hydrophobic drug carrying oil.

14. The ultrasound-responsive drug carrier of claim 8, wherein the inert gas includes at least part of perfluoromethane, perfluoroethane, perfluoropropane, perfluorobutane, perfluoropentane, perfluorohexane, perfluoroheptane, perfluorooctane, decafluoropentane, perfluoro (2-methyl-3-pentanone), perfluorotributylamine, perfluoro-15-crown-5-ether, perfluoro-1, 3-dimethylcyclohexane, perfluoromethylcyclopentane, perfluorodecalin, perfluoromethyldecalin, perfluoroperhydrobenzyltetralin, PERFECTA and sulfur hexafluoride.
